(19) United States
(12) Patent Application Publication
(10) Pub. No.: US 2022/0386892 A1
(43) Pub. Date: Dec. 8, 2022

(54) METHOD OF BREATH SCREENING OF VIRAL INFECTION

(71) Applicant: Teknoscan Systems, Inc., Vaughan (CA)

(72) Inventor: Sabatino NACSON, Thornhill (CA)

(21) Appl. No.: 17/824,702

(22) Filed: May 25, 2022

Related U.S. Application Data
(60) Provisional application No. 63/193,248, filed on May 26, 2021.

(30) Foreign Application Priority Data
May 25, 2021 (CA) 3119501

(51) Int. Cl. A61B 5/08; G01N 1/22; A61B 5/097

(52) U.S. Cl. CPC A61B 5/082; G01N 1/22; A61B 5/097; G01N 2001/2244

(57) ABSTRACT

An apparatus for detecting Covid-19 infection in a subject, the apparatus comprising (a) a sampling apparatus for collecting a breath sample from a subject (b) An analyzer, comprising an ion mobility spectrometer (IMS), for receiving the sample from the sampling apparatus and for determining the presence in the sample of Volatile Organic Compounds (VOCs) indicative of Covid-19, the VOCs comprising at least three compounds selected from the group consisting of C1-C3 alcohols, C2-C8 aldehydes, C3-C4 ketones and C4-C6 alkyl esters.

### RELATED APPLICATIONS

This application claims the benefit of U.S. Provisional Application No. 63/193,248, filed May 26, 2021, entitled “Method of Breath Screening of Viral Infection”, and claims the benefit of and priority to Canadian Application No. 3,119,501, filed May 25, 2021, which are hereby incorporated herein by reference in their entireties.

### FIELD OF THE INVENTION

This invention relates to the field of testing for COVID-19 infection in subjects.

### BACKGROUND OF INVENTION

The Covid-19 pandemic has created a need for large scale testing for cases of SARS-CoV-2, the virus that causes Covid-19. People requiring tests include the public at large, and also, longitudinal testing of high-risk sub-populations, such as health care workers.

COVID-19 testing is currently based on quantitative reverse transcription polymerase chain reaction (RT-qPCR) assays detecting SARS-CoV-2 RNA in nasopharyngeal (NP) and/or oral swabs. These tests: require authorized laboratories with a minimum Biological Safety Class 2 specification; take time to ship, process and report; and are prone to false negative results (from errors in swab sampling or laboratory processing, or because the virus is not yet/no longer present in the oropharynx). The false negative rate for one-time NP testing by RT-qPCR is 30% to 50% for COVID-19 samples acquired in community or clinical care settings, while the area-under-the-receiver operator characteristic (AUROC) for a single RT-qPCR test is about 0.8. Repeat RT-qPCR tests, combined with hematological variables and chest computed tomography, are advised for diagnosis, along with caution in the interpretation of negative RT-qPCR tests.

Exhaled breath analysis has attracted notable scientific and clinical interest in recent years. Volatile organic compounds (VOCs) have the potential to mirror various metabolic processes both locally within the respiratory system and systemically, via blood circulation. VOCs have been utilized as diagnostic, prognostic, and treatment response biomarkers for various respiratory illnesses, including infections.

U.S. Pat. Nos. 9,170,232 and 9,541,525 describe an ion-mobility spectrometer with front fast GC separation of the sample analytes. U.S. Pat. No. 9,329,156 describes a filter used in the collection of sample breath and enrichment of the sample. U.S. Pat. No. 5,395,589 discloses an apparatus for preconcentrating trace amounts of organic vapors in a sample of air for subsequent detection.

### SUMMARY OF THE INVENTION

It is desirable to provide a point-of-care testing apparatus and method because results can be obtained relatively quickly (preferably less than 10 minutes). Also, because there is no sample that needs to be transported for analysis, there is much reduced risk that it will be lost, damaged, or contaminated during sample collection, transport, and analysis.

It is also desirable to provide a non-invasive or less invasive form of COVID-19 testing. There are test subjects who have an aversion to more invasive types of sample collection instruments, such as swabs. This aversion can range from moderate to severe. Some test subjects will avoid testing completely, or at least suffer anxiety and discomfort from invasive sample collection. A non-invasive mode of sample collection can ameliorate these problems.

COVID-19 is a multi-system condition. It was hypothesized that a combination of inflammatory and host-response VOCs would differentiate between the breath of patients with COVID-19 and those with other respiratory or cardiac problems. After the start of the COVID-19 pandemic, independent feasibility studies were rapidly carried out with the following objectives: (1) to trial point-of-care testing using self-contained GC-IMS breath analyzers, and (2) to evaluate the breath biochemistry for possible markers of COVID-19.

Several studies have shown that specific VOCs related to disease are present as a result of contraction of that disease, and that some diseases can be identified using biomarkers(s) found in the subject's breath. Since COVID-19 eventually reaches the stage where it damages the lungs of the subject, it was hypothesized that some volatile and non-volatile biomarker(s), strongly related to the COVID-19 disease would be present in the alveolar breath and breath condensate as well as in body odour of the infected population that could be distinguished from the control (healthy) population.

In the biomarker discovery phase, a broad scope, untargeted investigation for volatile organic compounds (VOCs) found in breath and body scent of patients affected by COVID-19 was performed. Sample collection involved use of vapor enrichment cartridges (e.g., Tenax GC) with aspiration sample pump that can be used to screen a person's breath for several minutes. This allowed trapping and enrichment of VOCs exhaled from the patients. The intention was to screen and compare the sample of people who had tested positive and negative for COVID-19. The trapping cartridges were analysed at an accredited analytical laboratory using GC×GC separation and Time of Light Mass spectrometry (TOFMS) detection system. Chemometrics software with was used with the capability to extract maximum analytical information and identification of key components in samples of infected people compared to non-infected people. This facilitated discovery of differences between sample classes.

The disclosed invention relates in one aspect to collection media to trap exhaled breath from a person. Preferably, the subject being tested exhales five or more times and allowing enrichment of the exhaled air onto the collector media. The enriched breath sample is introduced into a short gas chromatography-ion mobility spectrometer for thermal desorption and analysis in a span of 20 seconds. The GC-plasmagram profile is analyzed by four-layer detection algorithm and an AI decision making process. The method was validated using COVID-19 infected subjects and healthy subjects. The invented method can be deployed at airports, shopping malls, office entrances, railway stations, universities, caregiver homes, etc. for rapid screening of the mass population. A non-invasive sampling approach of the method makes it a promising and fast technique for monitoring the health status of patients or volunteers during the vaccine trial and after vaccination.

### BRIEF DESCRIPTION OF THE DRAWINGS

Reference will now be made, by way of example only, to preferred embodiments of the invention and in which:

FIG. 1 is a representation of a breath sample being given.

FIG. 2 is a schematic representation of the preferred analyser system.

FIG. 3 is a typical 3D ion mobility spectrum or plasmagram 100 of a non-infected person.

FIG. 4 is a 3D ion mobility spectrum or plasmagram 200 of the breath of a COVID-19 infected person.

FIG. 5 is a schematic representation of the detector circuitry.

FIG. 6 is a schematic representation of alternative detector circuitry.

### DETAILED DESCRIPTION

In an aspect of the invention, a GC is used as a tool to separate out predetermined analytes, specifically volatile organic compounds (VOCs) whose presence has been found to be associated with Covid-19 infection.

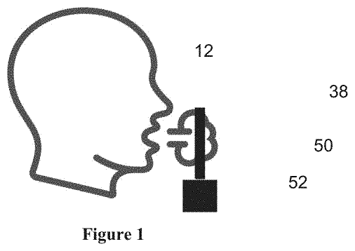

A sampling card 38 is illustrated in FIG. 1. The sampling card 38 comprises a substrate 50 coated with a combination of adsorbent/absorbent materials. The adsorbent/absorbent materials function as a chemical filter, concentrating vapors and entrapping fine airborne particles when air is directed over the sampling card 38. A handle 52 is formed at one end of the substrate 50. The handle 52 facilitates handling of the sampling card 38, allowing the sampling card to be readily inserted into and removed from a suitable sampler.

The substrate 50 may be formed of a stainless-steel mesh. Other possible substrate materials include nickel, copper, aluminum, fiberglass, porous Teflon, cotton, Nomex and other man-made fibers. Still other materials may be used. What is important is that materials be used that will retain the relevant VOCs for analysis.

The combination of adsorbent/absorbent materials may comprise two or more of diphenylene oxide polymer(s) prepared in chloroform, carbon composite materials such as graphite, fullerenes, polymeric carbons from soot produced from nitro substituted alkylbenzenes, divinyl benzene, mono-alkyl substituted benzenes, di-alkyl substituted benzene, toluene, xylenes, ethylbenzene, silicone oils with high thermal stability and boiling points and adsorption properties for wide range of organic compounds or other suitable materials, although silicone oils are not preferred.

VOCs on people's breath are usually present in the low parts per billion concentration and therefore, may well require some enrichment to bring the levels to the detection limit of the detector. The filter 50 is used to capture the volatile components on the infected person and excluding water and other light weight gases and preferably trap the target volatiles that indicate C OVID-19 infection.

The subject being tested will exhale, preferably ten times or more, onto the substrate 50, so that sample 12 comprising any relevant VOCs are absorbed into/adsorbed to the substrate material 50. Then the card 38, and the substrate 50, are inserted into heated desorber 14.

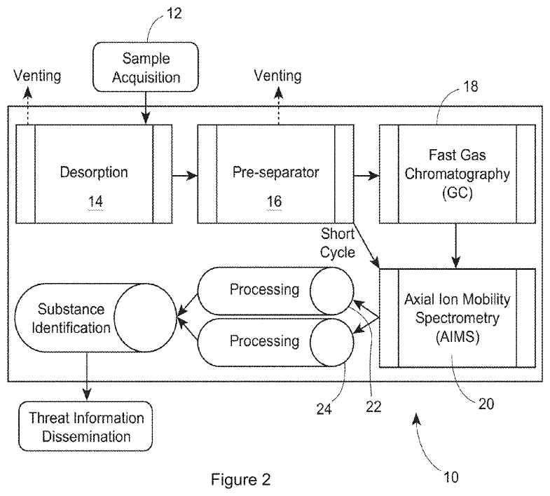

Referring now to FIG. 2, a schematic representation of the preferred analyser system 10, according to an aspect of the invention, is shown. The sample 12 is acquired by the analyser through interfacing with desorber 14. Desorber 14 communicates with pre-separator 16, which communicates both with GC 18, and AIMS 20. Processing means 22 and 24 are in communication with AIMS 20, and the outputs of means 22, 24 are used to identify substances of interest, after which identification information is disseminated. In the preferred embodiment, a carrier gas (discussed below) carries the sample from the desorber 14, to the pre-separator 16, the GC 18 and the AIMS 20.

Preferably, the desorber 14 includes means for ramping up temperature upon receipt of a sample to evaporate volatile compounds not of interest, thus cleaning the sample. These volatile contaminants are preferably vented. As the temperature continues to rise, the cleaned sample is then evaporated and travels to the pre-separator 16.

The desorber is heated to 200° C. which is sufficient to destroy any biological sample that is collected on the filter. Another aspect of the invention is trapping the effluent after internal sterilization on internal charcoal filter(s). This aspect of the invention makes the filter reusable for the next person.

Preferably, the desorber 14 communicates with the pre-separator 16 via a six-port heated valve, which functions to keep the sample evaporated until it condenses in the pre-separator 16. The pre-separator 16 is kept cool while the sample is transferred from the desorber 14, so that the sample will condense and thus be trapped.

The pre-separator 16 preferably operates as follows. It is heated in a ramping fashion with power pulses ranging from 100-500 μsec to assist in the thermal separation of different compounds based on their physical and chemical properties. Each compound will be released at a different temperature, and thus at a different time, creating a temporal separation between the individual predetermined analytes present. The pre-separator 16 also functions to release other volatile compounds not of interest that were not removed by the desorber 14, while separating in time the release of potential analytes of interest as the pulsed increase in temperature proceeds.

Thus, the desorber 14 and pre-separator 16 function to eliminate unwanted compounds and/or contaminants (such as volatile compounds), and thus to preselect for analysis compounds likely to be of interest.

Preferably, the pre-separated sample emerging from the pre-separator 16 is split into main and bypass samples. The bypass sample is carried directly to AIMS 20, permitting a faster analysis because of the GC step being skipped for the bypass sample. This faster analysis can, in the preferred embodiment, take about 20-30 seconds, providing a quick detection of threat substances followed by confirmation after GC analysis of the main sample is completed is completed. This offers flagging of the sample for further investigation.

On the other hand, if the short cycle shows no detection, there is a strong likelihood that the sample is clean. Preparations can begin to test the next sample. In the unlikely event that the long cycle shows detection when the short cycle did not, the relevant object (e.g., shipping containers, luggage, etc.) can be extracted and dealt with accordingly.

Preferably, the main sample is carried to the GC, and the preferred GC operates to evaporate the main sample by upward ramping of temperature. The main sample molecules are preferably trapped by adsorption, condensation, surface interaction on a cooled trapping material consisting of an inert coated metal surface like GC liquid phase and other means of trapping molecules. The trap is resistively heated by applying power across its terminals to release trapped materials into the carrier gas and transfer the evaporated main sample into the analytical GC column. The preferred GC column can contain polar, semi-polar or non-polar bonded liquid phase for effective separation of molecules of interest.

Temperature ramping of the preferred GC column under an internal carrier gas is accomplished by resistive heating of the column from 40 to 220 degrees Celsius, which allows separation of volatile and non-volatile (higher boiling point) compounds, typically in a span of 1-3 minutes. Positive and negative ions are formed for each analyte of interest, as well as dimer peaks because of the internal ion-molecular ionization processes. The initial temperature of the GC before heating is preferably maintained by an electrically driven cooling fan.

Those skilled in the art will appreciate that the analysis using the IMS 20 involves ionization, typically both positive and negative, of the sample entering the IMS. IMS devices, in general terms, identify analytes of interest by measuring mobility of associated ions using a drift tube and detector. Chemical ionization reagents (CIRs) are deployed in the IMS' ionization chamber to facilitate ionization of the substances in the sample for detection.

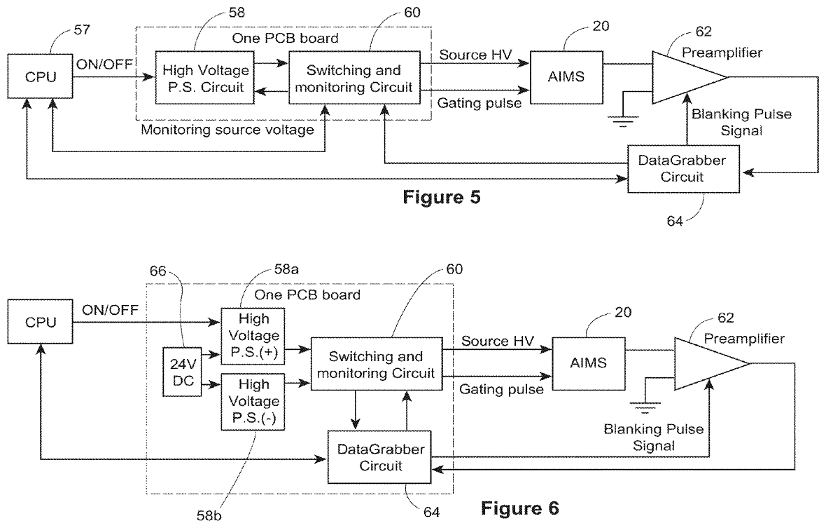

The preferred embodiment of the system is configured to time the deployment of CIRs to be concurrent with the GC peaks of analytes of interest. In the preferred embodiment, then, CIRs are conserved, and wastage reduced, since CIRs are deployed only when needed for ionization. In the preferred embodiment, the microprocessor controlling the system 10 is programmed to as to release CIRs to the IMS only concurrently with GC peaks, that is, when potential analytes of interest are arriving for analysis. CIRs are preferably withheld during the absence of GC peaks. Referring now to FIG. 5, the IMS assembly preferably comprises a microprocessor or CPU 57 which is configured to switch on and off high voltage power supply 58 (HVPS). HVPS 58 and CPU 56 are operatively connected to switching and monitoring circuit 60, which is used by CPU 56 to monitor the voltage from the HVPS and to switch the voltage.

The AIMS 20 receives the switching voltage and provides the raw output used to calculate ion mobility and identify, if appropriate, analytes of interest. The output is amplified by a pre-amplifier 62 prior to delivery to a data grabber circuit 64. It will be appreciated that the pre-amplifier is vulnerable to damage from sudden large changes in electric field resulting from changes in polarity and ionization of the sample. Specifically, damage may result from sudden change of voltages and voltage surge on the guard electrode located in front of the IMS' Faraday collector plate. The system 10 is thus configured to provide a protective blanking pulse signal to the pre-amplifier timed to coincide with the changes in the electric field, thus preventing the damage.

Circuit 60 preferably provides the high voltage polarity needed to operate the axial ion mobility spectrometer (AIMS) in one polarity and the appropriate gating pulse to introduce single polarity ions into the single glass or ceramic tube drift tube. The process is under CPU control. The signal generated at the preamplifier 62 is fed to the data grabber board 64 which controls the blanking pulse and feedback to the switching and monitoring circuit and to the CPU 56.

In the preferred embodiment, the circuit 60 comprises a half H instead of four H bridge, which offers a simpler and faster switching circuit capability over other configurations.

Alternation between ion polarities is preferably governed by a timing circuit of duration varying from 100-500 msec, depending on the eluting GC peak from the chromatography column. In this mode, several positive ion scans are collected in one polarity and several negative ion scans are collected in the opposite polarity mode. This is possible because the GC peak is wide enough, and the switching frequency high enough, to provide enough data points associated with a single GC peak, for both positive and negative polarities. Preferably, a time gap is afforded between each polarity to allow stabilization of reagent ions and baseline.

In an alternate embodiment shown in FIG. 6, there are instead two HVPSs, 58a and 58b, one set to output positive voltage, and the other negative. In this embodiment, supplies 58a and 58b may both draw power from a 24 VDC power supply 66. The power supplies 58a and 58b themselves do not switch polarity. Rather, the circuit 60 switches between one HVPS and the other. Preferably, the data grabber rate is 100 k samples/sec or down to 10 microseconds/sample for improved peak resolution. The advantage of two separate high voltage power supply is ability to adjust the polarity independently for each HVPS. Also switch time is reduced, because polarity does not switch—preferably, switch time is reduced as low as 500 microseconds.

It has been discovered that the presence of C1-C3 alcohols, C2-C8 aldehydes, C3-C4 ketones and/or C4-C6 alkyl esters on a subject's breath are indicative of COVID-19 infections. In an embodiment of the invention, if three or more of these compounds are present then a COVID-19 positive result is returned.

The following were found to be strongly indicative of COVID-19 infection: Ethyl butyrate (an ester); Propionaldehyde (aldehyde); 2-butanone (a ketone); Heptaldehyde (aldehyde); Octanal (aldehyde); Butyraldehyde (aldehyde). Each chemical is detected as protonated ion (MH⁺) and its dimer ion (M₂H⁺). The said protonated molecular ion can cluster with an internal CIR to form an additional ionic signature of the analyte of interest. Some of the substances form negative ions by clustering with internal CIRs. In an embodiment of the invention, a positive result is returned if three or more of these strongly indicative compounds are sensed. In another embodiment, the sensing of four of these strongly indicative compounds returns a positive result.

Each of these VOCs has a number of peaks in both positive and negative IMS modes, associated with different reduced mobilities, as per table 1 below. Preferably, COVID-19 positive subjects are identified by means of a combined result of four or more channels as programmed.

TABLE 1
COVID-19 substance VOC IMS channels as programmed

| C19 | | |
|---|---|---|
| Channel | | Chemical compound |
| Name | VOC | type/function group |
| C − 1 VOC | Ethyl Butyrate − channel | Ester |
| C + 1 VOC | Propionaldehyde + 1 channel; | Aldehyde |
| | 2-butonone + channel | Ketone |
| C + 2 VOC | Heptaldehyde + 1 channel | Aldehyde |
| C + 3 VOC | Butyraldehyde + 1 channel | Aldehyde |
| C + 4 VOC | Heptaldehyde + 2 channel | Aldehyde |
| C + 5 VOC | Octanal + 1 channel | Aldehyde |
| C + 6 VOC | Ethyl Butyrate + channel | Ester |
| C + 7 VOC | Butyraldehyde + 2 channel | Aldehyde |

In an aspect of the invention, there is provided a novel Retention-Time Separation-Analysis (RTCA) test-system casting substance-quantifiers as distinguishable nest-peaks. A Drift-Time-Peak-Separation (DTPS) technique was used for time-clustered structures. Another aspect of the invention is use of combined Derivative-Based-Retention-Time-Separation-Approach (DBRTA), which allowed identification of low signal-noise peaks over background baseline.

Complex-cluster benchmarks with 3-4 nested peaks residing in the analyte of interest identification area with relatively high peak intensity were addressed by this novel-architecture of multi-Shard detection designed to reduce misdetection working in dual-single polarity schemes. This advancement increased resolution of nested structures characteristics of sensed complex chemical compositions.

FIG. 3 shows a typical 3D ion mobility spectrum or plasmagram 100 of a non-infected person.

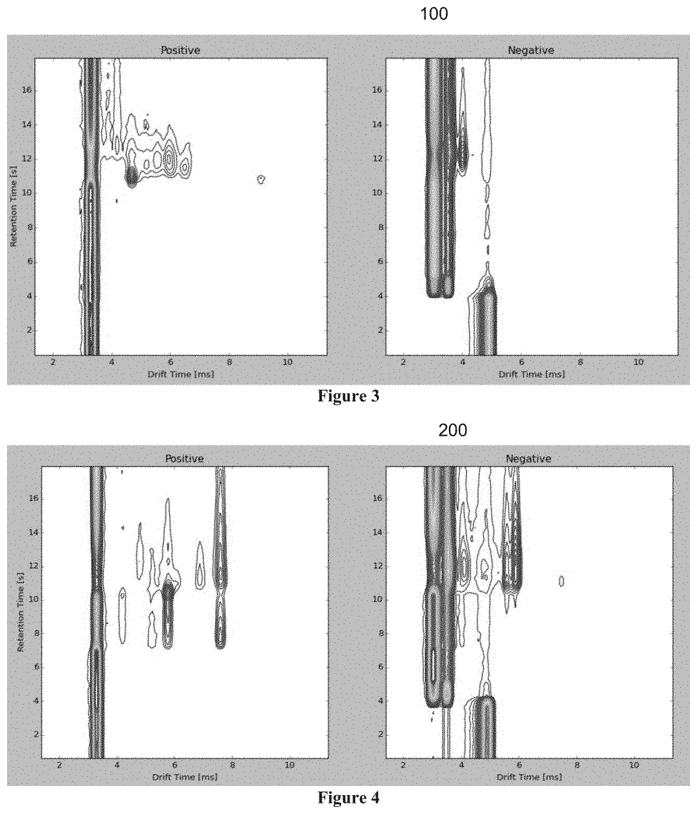

FIG. 4 shows a 3D ion mobility spectrum or plasmagram 200 of the breath of a COVID-19 infected person.

Embodiments of the invention include one or more of the following items.
1. Volatile organic compounds found on breath of infected subjects were identified and comprise of C1-C3 alcohols, C2-C8 aldehydes, C3-C4 ketones and C4-C6 alkyl esters.
2. A system as in item 1, wherein the system detects the ionic profile produced by the viral infection compared to breath samples from healthy people.
3. A system in item 2, wherein the VOCs are collected on a chemical treated filter. Five or more exhalation onto the filter to enrich the trapped VOCs.
4. A system as in item 1, wherein the temporal separation means comprises a pre-separator of the predetermined analytes and transfer into the chemical ionization source of the IMS.
5. A method of detecting the presence of plurality of predetermined analytes in the collected breath sample.
6. A method as in item 5, wherein the detected ionic species are protonated ions, dimers ions, analyte-cluster with chemical ionization reagent and negative ions clustering with oxygen and reagent ion.
7. A detector for detecting the presence of analytes profile using nested peaks within a non-Gaussian signal-pattern structure. Advanced compartmentalized Multi-Stacked Sharding with dynamic background-corrected noise identification algorithm.
8. A detector as in item 6, wherein the identification process uses multi-layer pattern recognition algorithm to identify the target analytes in the presence of complex chemical matrix.
9. An apparatus for detecting Covid-19 infection in a subject, the apparatus comprising:
   a. A sampling apparatus for collecting a breath sample from a subject;
   b. An analyzer, comprising an ion mobility spectrometer (IMS), for receiving the sample from the sampling apparatus and for determining the presence in the sample of Volatile Organic Compounds (VOCs) indicative of Covid-19, the VOCs comprising at least three compounds selected from the group consisting of C1-C3 alcohols, C2-C8 aldehydes, C3-C4 ketones and C4-C6 alkyl esters.

### REFERENCES

1. Russkiewicz et al, EClinical Medicine 100609, 2020. Diagnosis of COVID-19 by analysis of breath with gas chromatography-ion mobility spectrometry—a feasibility study.
2. Benjie Shan et all, ACS Nano. Aug. 18, 2020 and related references. Multiplexed nanomaterial-based sensor array for detection of COVID-19 exhaled breath.
3. M. Sohrab et al, Clin. Microbial, 3:3, 2014. Volatile organic compounds as novel markers for the detection of bacterial infections.
4. J. R. Belinato et al, J. Chrom. B, Volume 1110, Mar. 15, 2019. Rapid discrimination of fungal strains isolated from human skin based on microbial volatile organic profiles.
5. R. M. S. Thorn and J. Greenman, J. Breath Res. 6, 2012. Microbial volatile compounds in health and disease conditions.
6. A. A. El Qader et al, Biomed. Chromatogr. 29, 1783-1790, 2015. Volatile organic compounds generated by cultures of bacteria and viruses associated with respiratory infections.
7. B. Buszewski and T. L. igor, Anal. Bioanal. Chem, 404, 141-146, 2012. Identification of volatile lung cancer markers by GC-MS: comparison with discrimination by canines.
8. R. Jiang et al, Analytical Chimica Acta 804, 111-119, 2013. A non-invasive method for in vivo skin volatile compounds sampling.
9. P. Mochalski et al, J. Chrom. B, 1076, 29-34, 2018. Monitoring of selected skin and breath borne volatile organic compounds emitted from the human body using GC-IMS.
10. Maosheng Yao et al, https//doi.org/10.1101/2020.06.21.20136523. This version posted on Jun. 24, 2020. Breath-borne VOC Biomarkers for COVID-19.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Patents, patent publications and other references mentioned above in all sections of this application are herein incorporated by reference in their entirety for all purposes. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated.

1. An apparatus for detecting Covid-19 infection in a subject, the apparatus comprising:
a. a sampling apparatus for collecting a breath sample from a subject;
b. an analyzer, comprising an ion mobility spectrometer (IMS), for receiving the sample from the sampling apparatus and for determining the presence in the sample of Volatile Organic Compounds (VOCs) indicative of Covid-19, the VOCs comprising at least three compounds selected from the group consisting of C1-C3 alcohols, C2-C8 aldehydes, C3-C4 ketones and C4-C6 alkyl esters.

2. An apparatus as claimed in claim 1, wherein the group consists of Ethyl butyrate, Propionaldehyde, 2-butanone, Heptaldehyde, Octanal, and Butyraldehyde.

3. An apparatus as claimed in claim 1, wherein the IMS is programmed with a plurality of channels, and wherein the analyzer determines a positive result on at least three of the channels to determine a positive COVID-19 result, the channels comprising:

| C19 | | |
|---|---|---|
| Channel | | Chemical compound |
| Name | VOC | type/function group |
| C − 1 VOC | Ethyl Butyrate − channel | Ester |
| C + 1 VOC | Propionaldehyde + 1 channel; | Aldehyde |
| | 2-butonone + channel | Ketone |
| C + 2 VOC | Heptaldehyde + 1 channel | Aldehyde |
| C + 3 VOC | Butyraldehyde + 1 channel | Aldehyde |
| C + 4 VOC | Heptaldehyde + 2 channel | Aldehyde |
| C + 5 VOC | Octanal + 1 channel | Aldehyde |
| C + 6 VOC | Ethyl Butyrate + channel | Ester |
| C + 7 VOC | Butyraldehyde + 2 channel | Aldehyde |

4. An apparatus as claimed in claim 3, wherein the analyzer determines a positive result on at least four of the channels to indicate a positive COVID-19 result.

5. A method of testing for COVID-19 infection in a subject, the method comprising:
a. taking a breath sample from the person;
b. analysing the sample using an IMS-based analyser to determine whether at least three VOCs indicative of COVID-19 infection are present in the sample, the VOCs being selected from the group consisting of C1-C3 alcohols, C2-C8 aldehydes, C3-C4 ketones and C4-C6 alkyl esters; and
c. if at least three such VOCs are present in the sample, returning a positive result.

6. A method as claimed in claim 5, wherein the group consists of Ethyl butyrate, Propionaldehyde, 2-butanone, Heptaldehyde, Octanal, and Butyraldehyde.

7. A method as claimed in claim 5, wherein the IMS is programmed with a plurality of channels, and wherein the analyzer determines a positive result on at least three of the channels to determine a positive COVID-19 result, the channels comprising:

| C19 | | |
|---|---|---|
| Channel | | Chemical compound |
| Name | VOC | type/function group |
| C − 1 VOC | Ethyl Butyrate − channel | Ester |
| C + 1 VOC | Propionaldehyde + 1 channel; | Aldehyde |
| | 2-butonone + channel | Ketone |
| C + 2 VOC | Heptaldehyde + 1 channel | Aldehyde |
| C + 3 VOC | Butyraldehyde + 1 channel | Aldehyde |
| C + 4 VOC | Heptaldehyde + 2 channel | Aldehyde |
| C + 5 VOC | Octanal + 1 channel | Aldehyde |
| C + 6 VOC | Ethyl Butyrate + channel | Ester |
| C + 7 VOC | Butyraldehyde + 2 channel | Aldehyde |

8. A method as claimed in claim 7, wherein the analyzer determines a positive result on at least four of the channels to indicate a positive COVID-19 result.
